(19) United States
(12) Patent Application Publication
(10) Pub. No.: US 2026/0000814 A1
(43) Pub. Date: Jan. 1, 2026

(54) PROBIOTIC SELF-COATING MEDICAL CATHETER AND METHODS OF MAKING

(71) Applicant: Washington University, St. Louis, MO (US)

(72) Inventors: Hermann Frieboes, Louisville, KY (US); Jill Steinbach-Rankins, Redding, CT (US); Anthony Kyser, Louisville, KY (US); Nicole Gilbert, St. Louis, MO (US)

(21) Appl. No.: 19/252,433

(22) Filed: Jun. 27, 2025

Related U.S. Application Data
(60) Provisional application No. 63/664,946, filed on Jun. 27, 2024.

(51) Int. Cl. A61L 29/16; A61L 29/08; B33Y 70/00; C08G 77/20; C09D 11/102

(52) U.S. Cl. CPC A61L 29/16; A61L 29/085; C08G 77/20; C09D 11/102; A61L 2300/606; A61L 2420/02; B33Y 70/00

(57) ABSTRACT

Methods of making a probiotic self-coating medical catheter include preparing a probiotic bioink by combining at least one probiotic with a silicone bioink at a weight ratio of 2:1 to 100:1, printing a three-dimensional tubular structure with the probiotic bioink, and heating the three-dimensional tubular structure to a temperature of up to 60° C. to cure the probiotic bioink through hydrosilylation of the silicone bioink to form the probiotic self-coating medical catheter. The at least one probiotic is a non-pathogenic bacteria. A probiotic self-coating medical catheter made according to such method is also provided.

### CROSS-REFERENCE TO RELATED APPLICATIONS

This application claims priority to U.S. Provisional Application 63/664,946, filed Jun. 27, 2024, which is incorporated by reference herein in its entirety.

### STATEMENT REGARDING FEDERALLY SPONSORED RESEARCH OR DEVELOPMENT

This invention was made with U.S. Government support under Contract No. R01DK139034 by the National Institute of Health (NIH), National Institute of Diabetes and Digestive Kidney Diseases (NIDDK). The U.S. Government has certain rights in the invention.

### TECHNICAL FIELD

Embodiments of the present disclosure relate to medical catheters and more specifically medical catheters which are self-coating with a probiotic in vivo.

### BACKGROUND

Catheter infections, particularly catheter-associated urinary tract infections (CAUTIs), are a common concern in healthcare settings. These infections occur when bacteria or other microorganisms enter a patient's body through the catheter leading to potential complications like bacteremia and sepsis. Indwelling urinary catheters, which function to drain urine from bladders for extended periods of time are particularly problematic. However, the problem extends to other medical catheters including nasogastric tubes, intermittent urinary catheters, chest tubes, and other medical catheterization devices.

Catheters are commonly used to drain fluid from patients in the healthcare setting before, during, or after surgery. As a representative example, urinary catheters, which function to drain urine from bladders of patients who are unable to urinate naturally, are commonly used medical devices. In the healthcare setting, more than 30 million indwelling Foley catheters are used annually in the United States alone. Indwelling catheters are routinely placed for the period during and after surgical procedures and in long-term care patients with reduced mobility. Intermittent catheterization is widely used for bladder management in patients with neurogenic or idiopathic bladder dysfunction, urinary incontinence, or with spinal cord injuries. Global urinary catheter use is expected to increase due to increases in the rates of urinary incontinence and an overall aging global population.

Bladder catheterization, whether indwelling or intermittent, increases the risk of urinary tract infection. Catheter-associated urinary tract infections (CAUTI) account for 40% of nosocomial infections globally presenting a problem which is desirably overcome. Further, CAUTI are the leading source of secondary bloodstream infections, which can be life-threatening. A wide range of gram-negative bacteria such as Escherichia coli, Klebsiella pneumoniae and gram-positive bacteria such as Enterococcus, Staphylococcus aureus, Group B streptococcus as well as certain fungal species can cause CAUTI.

Urinary catheters increase the risk of UTI for several reasons. Insertion of the catheter through the urethra can result in translocation of periurethral bacteria into the bladder. The catheter surface serves as a site of microbial attachment where the invading CAUTI pathogen can form biofilms, which resist clearance by urine flow and are resistant to antibiotics.

There have been previous attempts to modify urinary catheters in order to reduce bacterial contamination, but such have proved ineffective at solving the ongoing problem. Antifouling agents alone on the catheter surface have been tried seeking to reduce attachment by steric or electrostatic hindrance. Attempts have also been made to coat or infuse catheters with biocidal agents, either contact-dependent or that elute from the surface, aim to kill invading pathogens.

### SUMMARY

As such, there is an ongoing need for improved prophylaxis in preventing catheter associated infections. The present disclosure introduces probiotic self-coating medical catheters which demonstrate advancements in efforts to prevent catheter associated infections. Generally, according to the present disclosure, the probiotic self-coating medical catheters as a result of the methods of making the same include at least one probiotic within a silicone matrix. The integration of the probiotic allows for self-coating of a probiotic bacterial interference layer on exposed surfaces of the probiotic self-coating medical catheters after in vivo placement. Bacterial interference approaches attempt to ward off infection by forming a coating on the catheter surface with nonpathogenic bacteria that can compete with invading pathogens. The idea behind this approach is that the presence of the nonpathogenic bacteria on the catheter surface will compete with invading pathogens.

Embodiments of the present disclosure relate to methods of making a probiotic self-coating medical catheter. The method includes preparing a probiotic bioink and printing a three-dimensional tubular structure with the probiotic bioink. Preparing the probiotic bioink includes combining at least one probiotic with a silicone bioink at a weight ratio of 2:1 to 100:1. Furthermore, at least one probiotic is a non-pathogenic bacteria. The method additionally includes heating the three-dimensional tubular structure to a temperature of up to 60° C., including embodiments at up to 50° C., to cure the probiotic bioink through hydrosilylation of the silicone bioink to form the probiotic self-coating medical catheter.

Embodiments of the present disclosure also relate to a probiotic self-coating medical catheter. The catheter includes a three-dimensional tubular structure having a central annulus, the tubular structure having a length, an outer diameter and an inner diameter with a wall thickness represented by the difference between the outer diameter and the inner diameter. Additionally, the tubular structure includes a probiotic bioink in a cured state where the probiotic bioink includes at least one probiotic and a silicone bioink at a weight ratio of 2:1 to 100:1.

### BRIEF DESCRIPTION OF THE DRAWINGS

The following detailed description of specific embodiments of the present disclosure can be best understood when read in conjunction with the following drawings.

FIG. 1 illustrates a three-dimensional tubular structure forming a probiotic self-coating medical catheter, according to embodiments herein.

FIG. 2 illustrates a graph of volume and outer diameter change of a comparative bioprinted scaffold and a bioprinted scaffold according to embodiments herein.

FIG. 3 illustrates a graph of mass loss in an artificial urine media of a comparative bioprinted scaffold and a bioprinted scaffold according to embodiments herein.

FIG. 4 illustrates a graph of daily release of probiotic in MRS of a comparative bioprinted scaffold and a bioprinted scaffold according to embodiments herein.

FIG. 5 illustrates a graph of daily release of probiotic in urine of a comparative bioprinted scaffold and a bioprinted scaffold according to embodiments herein.

FIG. 6 illustrates a graph of cumulative release of probiotic in MRS of a comparative bioprinted scaffold and a bioprinted scaffold according to embodiments herein.

FIG. 7 illustrates a graph of cumulative release of probiotic in urine of a comparative bioprinted scaffold and a bioprinted scaffold according to embodiments herein.

FIG. 8 illustrates proliferation of probiotic with incubation in MRS for a comparative bioprinted scaffold and a bioprinted scaffold according to embodiments herein.

FIG. 9 illustrates inhibition and killing of pathogenic bacteria in MRS for a comparative bioprinted scaffold and a bioprinted scaffold according to embodiments herein.

FIG. 10 illustrates inhibition and killing of pathogenic bacteria in MRS for a preincubated comparative bioprinted scaffold and a preincubated bioprinted scaffold according to embodiments herein.

FIG. 11 illustrates adherence of pathogenic bacteria to a comparative bioprinted scaffold and a bioprinted scaffold according to embodiments herein.

Reference will now be made in greater detail to various embodiments of the present disclosure, some embodiments of which are illustrated in the accompanying drawings.

### DETAILED DESCRIPTION

Embodiments of the present disclosure generally relate to a probiotic self-coating medical catheter and well as making the same. More specifically, the present disclosure incorporates at least one probiotic with a silicone bioink to form medical catheters which leverage bacterial interference approaches to prophylactically ward off infection by forming a coating on the catheter surface with nonpathogenic bacteria that can compete with invading pathogens.

Specific embodiments of the present application will now be described. The disclosure may, however, be embodied in different forms and should not be construed as limited to the embodiments set forth in this disclosure. Rather, these embodiments are provided so that this disclosure will be thorough and complete, and will fully convey the scope of the subject matter to those skilled in the art.

Embodiments of the present disclosure are directed to methods of making a probiotic self-coating medical catheter. The method includes preparing a probiotic bioink, in which preparing the probiotic bioink includes combining at least one probiotic with a silicone bioink at a weight ratio of 2:1 to 100:1. The at least one probiotic is a non-pathogenic bacteria to provide bacterial interference of pathogenic bacteria and/or fungi. The method additionally includes printing a three-dimensional tubular structure with the probiotic bioink. The three-dimensional tubular structure is then heated to a temperature of up to 60° C. to cure the probiotic bioink through hydrosilylation of the silicone bioink to form the probiotic self-coating medical catheter.

Embodiments of the present disclosure are also directed to probiotic self-coating medical catheters formed in accordance with the methods of the present disclosure. The probiotic self-coating medical catheters comprise a three-dimensional tubular structure having a central annulus, the tubular structure comprising a length, an outer diameter and an inner diameter with a wall thickness represented by the difference between the outer diameter and the inner diameter. Further, the tubular structure comprises a probiotic bioink in a cured state, the probiotic bioink comprising at least one probiotic and a silicone bioink at a weight ratio of 2:1 to 100:1.

The present disclosure provides novel bacterial interference strategies for inhibition of CAUTI and other catheter associated infections through 3D bioprinting. Extrusion-based 3D bioprinting enables incorporating viable cells into a formulated ink for manufacturing well-defined constructs for biomedical applications.

Having generally described the probiotic self-coating medical catheters and method of making the same in accordance with the present disclosure, each of the various components and processing steps will be provided in further detail.

### Bioprinting

Bioprinting techniques allow for the precise patterning of living cells into biocompatible materials in a pre-defined manner. The process of bioprinting includes preparation of the bioink for utilization in the bioprinting, printing of the desired article geometry, and post-printing activities. Prior to printing, the bioink is prepared. In accordance with the present disclosure, the bioink is a combination of silicone bioink and at least one probiotic to form a probiotic bioink used for bioprinting. Printing the desired article geometry includes printing a scaffold according to a model and cross-linking the scaffold, and the post-printing activity includes further crosslinking of the scaffold.

### Probiotic

The at least one probiotic included within the probiotic self-coating medical catheter is selected to provide bacterial interference against proliferation of pathogenic bacteria and/or fungi on the catheter surfaces. The probiotic may be any bacteria which does not harm the patient into which the probiotic self-coating medical catheter. Harm to the patient may include illness, a clinically significant immune response, or other harmful clinical responses or outcomes known to those skilled in the art. The probiotic may also be selected to be selected to be a bacteria endogenous to the placement site of the probiotic self-coating medical catheter. While the probiotic may be broadly selected in accordance with various embodiments of the present disclosure, for conciseness and clarity focus will be directed to Lactobacillus genera.

In accordance with one or more embodiments, the at least one probiotic is from the genus Lactobacillus. Representative Lactobacillus species within the Lactobacillus genera include, without limitation, Lactobacillus acidophilus (L. acidophilus), Lactobacillus rhamnosus (L. rhamnosus), Lactobacillus gasseri (L. gasseri), Lactobacillus reuteri (L. reuteri), Lactobacillus bulgaricus (L. bulgaricus), Lactobacillus plantarum (L. plantarum), Lactobacillus johnsonii (L. johnsonii), Lactobacillus paracasei (L. paracasei), Lactobacillus casei (L. casei), Lactobacillus crispatus (L. crispatus), and Lactobacillus salivaris (L. salivaris).

Selection of Lactobacillus probiotics to provide the bacterial interference strategy to prevent CAUTI and other catheter associated infections provides overall benefits. Lactobacillus bacterial are already a part of the beneficial human bacterial flora. For example, lactobacilli are the most abundant members of the human vaginal and urinary microbiomes and are associated with healthy balance in both body sites. Lactobacillus is associated with positive benefits based on its presence in the human vaginal and urinary microbiomes. For example, beneficial vaginal health outcomes, including protection from acquisition of sexually transmitted infections and bacterial vaginosis have been attributed to the presence of lactobacilli. Additionally, during pregnancy, women with a vaginal microbiome dominated by lactobacilli are less likely to have preterm birth or other adverse pregnancy outcomes. Urogenital Lactobacillus may also act to protect against colonization or infection with uropathogens.

Selection of Lactobacillus probiotics to provide the bacterial interference strategy to prevent CAUTI and other catheter associated infections is also believed beneficial as a strong antimicrobial activity of Lactobacillus probiotics against a wide range of bacterial and fungal uropathogens relevant to CAUTI and other catheter associated infection, including uropathogenic E. coli has been demonstrated in vitro. Probiotic strains of Lactobacillus and Lactobacillus strains isolated from the urogenital microbiome kill pathogens with a variety of mechanisms including production of lactic acid, hydrogen peroxide, and bacteriocins. These mechanisms are similar to the endogenous Lactobacillus in the microbiome. Therefore, locally administered Lactobacillus probiotics with development of a coating of the probiotic on the self-coating medical catheter is believed capable of killing invading pathogens without disrupting the endogenous Lactobacillus in the microbiome. Additionally, as previously noted Lactobacillus strains can form biofilms and thus limit pathogen access to the catheter surface providing the desired bacterial interference.

Selection of Lactobacillus probiotics to provide the bacterial interference strategy to prevent CAUTI and other catheter associated infections as species within the Lactobacillus genus have been demonstrated to be clinically safe and tolerated within a human bladder. Specifically, L. rhamnosus GG has been in use for more than three decades, is widely studied and well-characterized, with demonstrated safety in the human urinary tract. Therefore, if an individual has a dysbiotic microbiome such as CAUTI, the presence of Lactobacillus provided with the self-coating medical catheter would be expected to help restore urogenital health by displacing the dysbiotic microflora.

### Silicone Bioink

Bioinks generally are a combination of one or more polymers. The polymers used in bioinks typically are chosen based on their specific properties. There are a number of different types of bioinks. For example, there are matrix bioinks, which are cell-laden mixtures used to shield the cells from the shear stress of printing, sacrificial bioinks, which are materials that can be removed after printing, and support bioinks, which are materials with specific mechanical properties. The bioinks of the present disclosure are formulated to deliver the one or more probiotics as part of the probiotic self-coating medical catheter.

In accordance with one or more embodiments of the present disclosure, the bioink is formulated to form a probiotic bioink formed from a silicone bioink with the at least one probiotic as previously addressed intermixed. In one or more embodiments, the silicone bioink comprises polydimethylsiloxane. Specifically, the silicone bioink may be formulated as a copolymer of the silicone bioink comprises a copolymer of vinyl-terminated poly(dimethylsiloxane) and vinyl, methyl-modified silica as part A and methylhydrosiloxane-dimethylsiloxane copolymer, trimethylsiloxane-terminated as part B. In various embodiments, vinyl-terminated poly(dimethylsiloxane) and vinyl, methyl-modified silica provided as part A may be provided at a weight ratio of 60:40 to 80:20, 65:35 to 80:20, 70:30 to 80:20, 60:40 to 75:25, 65:35 to 75:25, 70:30 to 75:25, 60:40 to 70:30, 65:35 to 70:30, or approximately 70:30. In various embodiments, part A and part B may be provided at a weight ratio of 5:1 to 15:1, 7:1 to 15:1, 9:1 to 15:1, 5:1 to 12:1, 7:1 to 12:1, 9:1 to 12:1, 5:1 to 11:1, 7:1 to 11:1, 9:1 to 11:1, or approximately 10:1 to form the silicone bioink.

### Probiotic Bioink

In accordance with one or more embodiments of the present disclosure, probiotic bioink is prepared. The probiotic bioink comprises the at least one probiotic with the silicone bioink. According to various embodiments, the at least one probiotic may be combined with the silicone bioink at a weight ratio of 2:1 to 100:1, 2:1 to 80:1, 2:1 to 60:1, 2:1 to 40:1, 2:1 to 20:1, 2:1 to 15:1, 4:1 to 100:1, 4:1 to 80:1, 4:1 to 60:1, 4:1 to 40:1, 4:1 to 20:1, 4:1 to 15:1, or approximately 4:1. The formulation of the probiotic bioink may alternatively be provided, in accordance with one or more embodiment, at a loading of 10⁶ to 10⁹ cfu (colony forming units) of the probiotic per milligram (mg) of silicone bioink. In accordance with various further embodiments, the probiotic bioink may be provided at a loading of 10⁶ to 10⁸, 10⁶ to 10⁷, 10⁷ to 10⁹, 10⁷ to 10⁸, or about 10⁷ cfu per mg of silicone bioink.

In one or more embodiments, the one or more probiotics may be added to the silicone bioink and mixed to form the probiotic bioink. Alternatively, in one or more embodiments, the silicone bioink may be added to the one or more probiotics and mixed to form the probiotic bioink. It is noted that upon preparation of the silicone bioink crosslinking and curing of the silicone bioink naturally progresses so mixing with the one or more probiotics should be completed in a time sensitive manner to allow for subsequent bioprinting with the generated probiotic bioink.

In one or more embodiments, the copolymers forming the silicone bioink and the one are more probiotics are combined concurrently and before formulation of a complete silicone bioink. For example, in one or more embodiments, vinyl-terminated poly(dimethylsiloxane) and vinyl, methyl-modified silica along with methylhydrosiloxane-dimethylsiloxane copolymer, trimethylsiloxane-terminated are combined with L. rhamnosus to generate the probiotic bioink.

In one or more embodiments, the probiotic bioink additionally comprises at least one antibiotic. Specifically, preparing the probiotic bioink comprises combining at least one probiotic, the silicone bioink, and at least one antibiotic. The at least one antibiotic may be any medication used to treat and prevent bacterial infections in humans and animals and may be selected to be effective at killing or stopping growth and multiplication of one or more specific bacteria. In various embodiments, the at least one antibiotic may be amoxicillin, amoxicillin-clavulanate, cephalexin, cefadroxil, nitrofurantoin, trimethoprim-sulfamethoxazole, ciprofloxacin, levofloxacin, Fosfomycin, or their combinations in clinically relevant dosages.

In one or more embodiments, the probiotic bioink additionally comprises at least one composite filler. Specifically, preparing the probiotic bioink comprises combining the at least one probiotic, the silicone bioink, and at least one composite filler. The at least one composite filler may be any biocompatible material. In various embodiments, the at least one composite filler may be particles or strands of ceramic, silica, polymer, or their combinations. In one or more embodiments, polymers used as the composite filler may be limited to polymers having a molecular weight of 50 kilodalton (kDa) or greater.

### Bioprinting

Various 3D printing techniques that are suitable and can be used to fabricate probiotic self-coating medical catheters described herein. It will be appreciated that the inclusion of the at least one probiotic limits 3D printing techniques to those that don't require chemical solvents or high temperatures as part of the printing or curing process. Specifically, 3D printing techniques which require chemical solvents or high temperatures may kill, deactivate, or otherwise have a detrimental effect on the viability of the at least one probiotic. Example 3D printing techniques include extrusion and co-axial extrusion, fused deposition modelling, inkjet bio-printing, laser-assisted bioprinting, stereolithography, selective laser sintering (SLS), or combinations thereof. It would be appreciated that essentially any 3D bioprinting process can be used that allows for the ability to print an aqueous solution at a temperature and pressure that is not harmful to the cells.

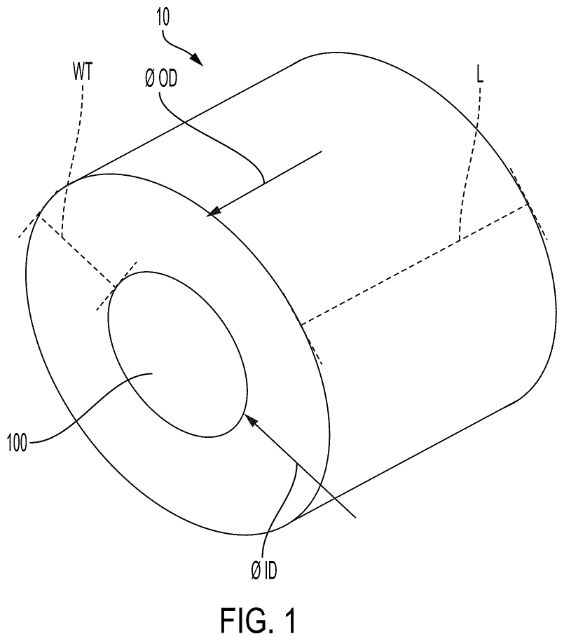

In accordance with embodiments of the present disclosure, the 3D printing technique is utilized to print a three-dimensional tubular structure with the probiotic bioink. The three-dimensional tubular structure forms the shape and geometry of the probiotic self-coating medical catheter. With reference to FIG. 1, an example three-dimensional tubular structure 10 is illustrated. The three-dimensional tubular structure 10 comprises a central annulus 100. Additionally, the tubular structure comprises a length (L), an outer diameter (OD) and an inner diameter (ID) with a wall thickness (WT) represented by the difference between the outer diameter and the inner diameter. The length, outer diameter, and inner diameter of the three-dimensional tubular structure 10 are selected to generate the probiotic self-coating medical catheter in conformity with clinically significant dimensions. For clarity, it is noted that medical catheters are commonly sized using the French (Fr) scale where 1 Fr equals 0.33 mm in (external) diameter. Embodiments, of the present disclosure may include catheters ranging from 1 Fr to 30 Fr.

In one or more embodiments, the probiotic self-coating medical catheter is configured as a urinary catheter. Specifically, the length, outer diameter, and inner diameter of the three-dimensional tubular structure 10 are selected to generate the probiotic self-coating medical catheter with dimensions aligned with those of a urinary catheter such that the probiotic self-coating medical catheter may be utilized as a urinary catheter in clinical applications. Accordingly, in various embodiments, the probiotic self-coating medical catheter may be a 6 Fr to 26 Fr catheter having an outer diameter or bore size of 2 mm to 8.7 mm, a wall thickness of 0.3 mm to less than half the outer diameter to maintain the central annulus 100, and a length of 9 to 46 cm. It will be appreciated that further sizes may be utilized to align with the full breadth of urinary catheters desired in clinical applications. Accordingly, it is expressly noted that the probiotic self-coating medical catheter may have various dimensions to align with the standard sizing, typically Fr scale sizing, utilized clinically for urinary catheters.

In one or more embodiments, the probiotic self-coating medical catheter is configured as a nasogastric tube. Specifically, the length, outer diameter, and inner diameter of the three-dimensional tubular structure 10 are selected to generate the probiotic self-coating medical catheter with dimensions aligned with those of a nasogastric tube such that the probiotic self-coating medical catheter may be utilized as a nasogastric tube in clinical applications. Accordingly, in various embodiments, the probiotic self-coating medical catheter may be a 3.5 Fr to 18 Fr catheter having an outer diameter of 1 mm to 6 mm, a wall thickness of 0.3 mm to less than half the outer diameter to maintain the central annulus 100, and a length of 10 to 200 cm. It will be appreciated that further sizes may be utilized to align with the full breadth of nasogastric tubes desired in clinical applications. Accordingly, it is expressly noted that the probiotic self-coating medical catheter may have various dimensions to align with the standard sizing, typically Fr scale sizing, utilized clinically for nasogastric tubes.

In one or more embodiments, the probiotic self-coating medical catheter is configured as an intrapleural chest tube. Specifically, the length, outer diameter, and inner diameter of the three-dimensional tubular structure 10 are selected to generate the probiotic self-coating medical catheter with dimensions aligned with those of an intrapleural chest tube such that the probiotic self-coating medical catheter may be utilized as an intrapleural chest tube in clinical applications. Accordingly, in various embodiments, the probiotic self-coating medical catheter may be a 6 Fr to 40 Fr catheter having an outer diameter of 2 mm to 13.3 mm, a wall thickness of 0.3 mm to less than half the outer dimeter to maintain the central annulus 100, and varying lengths. It will be appreciated that further sizes may be utilized to align with the full breadth of intrapleural chest tubes desired in clinical applications. Accordingly, it is expressly noted that the probiotic self-coating medical catheter may have various dimensions to align with the standard sizing, typically Fr scale sizing, utilized clinically for intrapleural chest tubes.

It is noted that while various dimensions, including lengths, are provided for example applications of the probiotic self-coating medical catheter, the length of the three-dimensional tubular structure may be provided outside the disclosed lengths. Specifically, the three-dimensional tubular structure may be prepared to generate a length of medical catheter sufficient to extend outside a patient at which point a medical catheter of conventional materials and manufacture may be joined for further extension. For example, the three-dimensional tubular structure for an intrapleural chest tube may be prepared with sufficient length to extend beyond the chest cavity to provide the prophylaxis afforded by the integrated probiotic with an additional medical catheter provided to extend the total length of the intrapleural chest tube. Additionally, it is noted that the three-dimensional tubular structure may be printed with the probiotic bioink for only a portion of the length with the silicone bioink or a bioink of alternate formulation being utilized to print the remainder of the three-dimensional tubular structure. Such allows the probiotic to only be included in portion of the probiotic self-coating medical catheter which are susceptible to pathogenic bacteria infiltration or coating.

The probiotic self-coating medical catheters in accordance with the present disclosure may be indwelling or intermittent catheters.

In accordance with embodiments of the present disclosure, the probiotic bioink forming the three-dimensional tubular structure 100 is cured to generate the probiotic self-coating medical catheter. In various embodiments, the three-dimensional tubular structure 100 is heated to a curing temperature of up to 60° C., up to 58° C., up to 56° C., up to 54° C., up to 52° C., or up to 50° C. to cure the probiotic bioink. Additionally, in various embodiments, the three-dimensional tubular structure 100 is heated to a curing temperature of at least 25° C., at least 30° C., at least 35° C., at least 40° C., at least 45° C., or at least 50° C. bounded by the previously disclosed upper bounds to controllably cure the probiotic bioink. In one or more embodiments, curing the probiotic bioink is achieved through hydrosilylation of the silicone bioink, thereby forming the probiotic self-coating medical catheter. It will be appreciated that capping the curing temperature alleviates destruction of viability of the probiotic in the probiotic bioink. Specifically, heating the probiotic above a threshold temperature results in increased or complete kill off of the bacteria forming the probiotic, thereby negating any benefit and destroying the self-coating nature of the probiotic self-coating medical catheter. The specific threshold temperature at which the viability of the probiotic is diminished is dependent upon the specific bacterial species and strain forming the probiotic.

In accordance with various embodiments, the three-dimensional tubular structure 100 may be held at the curing temperature for 6 to 48 hours, 12 to 48 hours, 18 to 48 hours, 24 to 48 hours, 6 to 36 hours, 12 to 36 hours, 18 to 36 hours, 24 to 36 hours, 6 to 24 hours, 12 to 24 hours, 18 to 24 hours, or approximately 24 hours to achieve sufficient cross-linking of the probiotic bioink in the three-dimensional tubular structure 100.

### Examples

To demonstrate the feasibility and utility of forming the probiotic self-coating medical catheters of the present disclosure, test specimens were generated for biological and mechanical testing.

### Probiotic Bioink and Bioprinting

As previously disclosed, the probiotic bioink is formed by combining at least one probiotic with a silicone bioink. Accordingly, the probiotic and the silicone bioink are necessarily grown, generated, or procured.

For the test specimens, the selected probiotic was L. rhamnosus. Accordingly, L. rhamnosus was cultured on De Man, Rogosa, and Sharpe (MRS). MRS medium is designed to encourage the growth of Lactobacilli while suppressing the growth of other bacteria, particularly Gram-negative bacteria. The MRS medium was supplemented with 0.1% Tween 80. The culturing was completed on agar plates under anaerobic conditions at 37° C. After 48 h, colony formation was visually evident on the agar plates. L. rhamnosus was then subcultured by selecting a single colony from the agar plates and additional culturing in MRS broth was completed in a closed tube at 37° C. for an additional 24 h. Subsequent subcultures were established by diluting 200 μL of L. rhamnosus with 9.8 mL of MRS broth, representing a 1:50 dilution.

To generate the probiotic bioink for the test specimens, the generated L. rhamnosus subculture was quantified. Specifically, a Nanodrop 2000 (Thermo Scientific, MA) was utilized to measure the absorbance value (x=OD600) to determine the volume of L. rhamnosus solution (y) to add when formulating the probiotic bioink, using the equation y=((9×10⁷)×(x))−2×10⁷ to obtain a theoretical loading of 5×10⁷ colony forming units (CFU) per mg of the silicone bioink. The necessary volume of L. rhamnosus solution was centrifuged at 3500 g for 10 minutes. The supernatant was removed and the L. rhamnosus pellet was transferred to a new centrifuge tube.

The probiotic bioink for the test specimens was formulated from a mixture of silicone and L. rhamnosus. The silicone bioink was formulated with a 10:1 weight ratio of vinyl-terminated poly(dimethylsiloxane) (70%) and vinyl, methyl-modified silica (30%):methylhydrosiloxane-dimethylsiloxane copolymer, trimethylsiloxane-terminated. The silicone bioink was added to the centrifuge tube containing the L. rhamnosus pellet at a 4:1 w/w ratio. The components were blended together in a biosafety cabinet to maintain sterility until the mixture became a visually uniform paste. The resulting probiotic bioink was transferred into a compatible bioprinter syringe for bioprinting.

The Allevi 3 Bioprinter (3D Systems, Rock Hill, SC) was used to bioprint scaffolds for the test specimens. The CORE head of the Allevi 3 Bioprinter was heated to 37° C., and then 1 mL of L. rhamnosus-containing bioink, or silicone bioink alone without any L. rhamnosus for blank control specimens, was loaded into the syringe and placed into the CORE head. To attain fine printing resolution, the printing rate was set at 5 mm/s with an extrusion pressure set at 100 psi. Each printing slice was printed at a thickness of 0.2 mm and an infill distance of 0.35 mm was utilized. The scaffolds were printed to generate a three-dimensional tubular structure according to the present disclosure with a length of 3 mm, outer diameter of 4 mm, and an inner diameter of 1.85 mm. Specimens created with the L. rhamnosus-containing bioink (probiotic bioink) represent Inventive Sample 1 and specimens created with silicone bioink alone represent Comparative Sample 2. The resulting bioprints were cured for 24 h at 50° C.

### Volume and Degradation Stability

It will be appreciated that medical catheters are specifically sized to meet clinical needs including catheter dimensions and flow rates through the central annulus. Additionally, medical catheters, such as urinary catheters, are often placed in patients for extended periods of time; therefore, it is important that they maintain their shape and physical integrity. Accordingly, the stability of the dimensions of the test specimens of Inventive Sample 1 and Comparative Sample 2 were quantified. Additionally, as medical catheters may be in place for an extended period of time, mass degradation of the test specimens was also quantified.

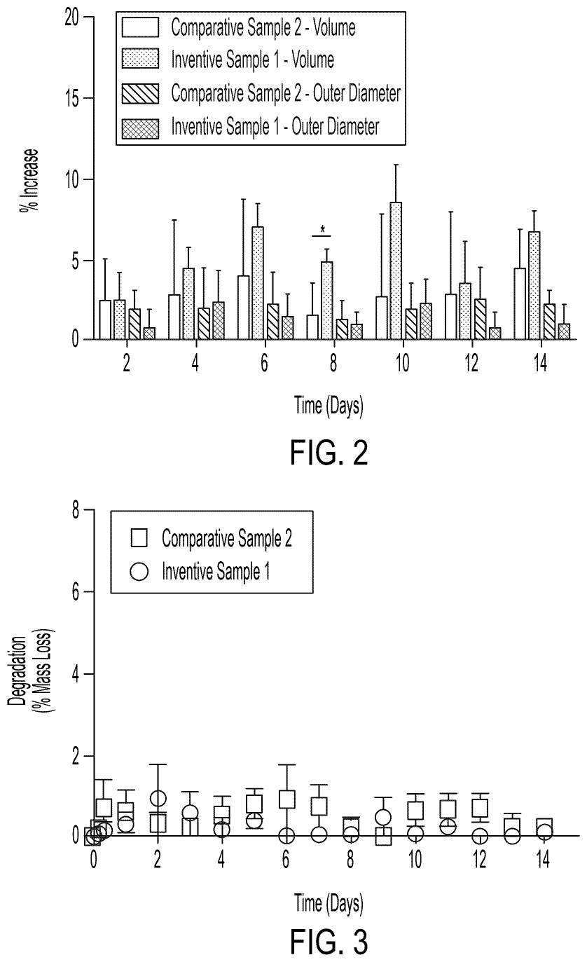

To quantify change in test specimen dimensions, each of Inventive Sample 1 and Comparative Sample 2 were initially measured with calipers to determine the initial volume prior to immersion in an artificial urine media (AUM). The AUM comprised calcium chloride hydrate (Sigma, 202940), magnesium chloride (Sigma, 1374248), sodium chloride (Sigma, 746398), sodium sulfate (Sigma, 239313), sodium citrate (EM Science, SX0445-3), sodium oxalate (Sigma, 379735-5G), urea (Alfa Aesar, A12360), monopotassium phosphate (Sigma, P0662), potassium chloride (Fisher, P217), ammonium chloride (Fisher, MFCD00011420), creatine (Sigma, C0780-50G), and tryptic soy broth (Sigma, 22092-500G). At time points of 2 days, 4, days, 6 days, 8 days, 10 days, 12 days, and 14 days after immersion into the AUM, each of Inventive Sample 1 and Comparative Sample 2 were measured with calipers to determine the change in volume and outer diameter corresponding with the swelling at each time point. FIG. 2 graphically illustrates the percent increase in volume and outer diameter for each specimen at each time point. FIG. 2 demonstrates a minimal increase in volume and outer diameter as a result of placement in an AUM media. Specifically, volume increase was less than 10% of the initial volume, and outer diameter increase was minimal (<3%) within this time period, showing minimal change. Accordingly, it is believed that probiotic self-coating medical catheters prepare in accordance with the present disclosure would substantially retain clinically appropriate dimensions.

To quantify mass degradation for each of Inventive Sample 1 and Comparative Sample 2 in a simulated environment for a urinary catheter, the mass loss of a representative sample of each of Inventive Sample 1 and Comparative Sample 2 was measured at 4 hours, 8 hours, and each day for 14 days. The mass of each of Inventive Sample 1 and Comparative Sample 2 were initially measured after curation (Wi) and then immersed in 1 mL of AUM for the 4 hours, 8 hours, and 1-14 days. At each time point one representative sample of Inventive Sample 1 and Comparative Sample 2 was removed from the AUM and dried at 50° C. for 12 h to ensure full dryness. A final weight of the samples (Wi) was measured. Accordingly, the mass loss percentage was determined according to equation 1.

$$mass degradation percentage = [ ( WiWf ) / Wi ) ] \times 100 \qquad Equation 1$$

FIG. 3 graphically illustrates the % mass loss for each specimen at each time point. FIG. 3 demonstrates a degradation of the samples. The change in Inventive Sample 1 with L. rhamnosus mass was less than 2% of its initial value over 14 days and had no significant difference compared to the L. rhamnosus-free Comparative Sample 2. Accordingly, it is believed that probiotic self-coating medical catheters prepare in accordance with the present disclosure would substantially retain clinically appropriate stability.

### Mechanical Integrity

It will be appreciated that medical catheters are subjected to compression and tension while in place and during insertion or removal. Specifically, insertion and removal of urinary catheters exerts mechanical stress on the catheter material. Incorporation of live bacteria could affect the mechanical strength of bioprinted catheters. Therefore, mechanical properties of the formulated biomaterial were assessed through uniaxial tensile testing. Accordingly, the mechanical integrity, and more specifically the tensile strength and compressive load strength of structures generated according to methods of the present disclosure were tested. Tensile strength and load resistance were measured through tensile testing. Testing samples having a dog bone shape in accordance with ASTM D412 standard dimensions were prepared using silicone bioink without L. rhamnosus and with probiotic bioink containing L. rhamnosus as formulated for Inventive Sample 1 and Comparative Sample 2. The maximum load stress of each material was measured using a MTI-2K-mini testing system. Tensile testing was performed using a 50 kN load cell at a velocity of 500 mm/min until fracture. The silicone bioink samples without L. rhamnosus had an average tensile strength of 7.28 MPa and maximum resistance to a load of 8.98 N. The probiotic bioink sample containing L. rhamnosus exhibited a tensile strength of 2.2 MPa with maximum resistance to a load of 2.3 N. These values are aligned with those of aged Foley catheters which have demonstrated tensile strength of 2 to 5 MPa, and the strength of new 12 Fr latex urinary catheters which have a tensile strength of 2.7 MPa.

### Probiotic Recovery and Catheter Coating

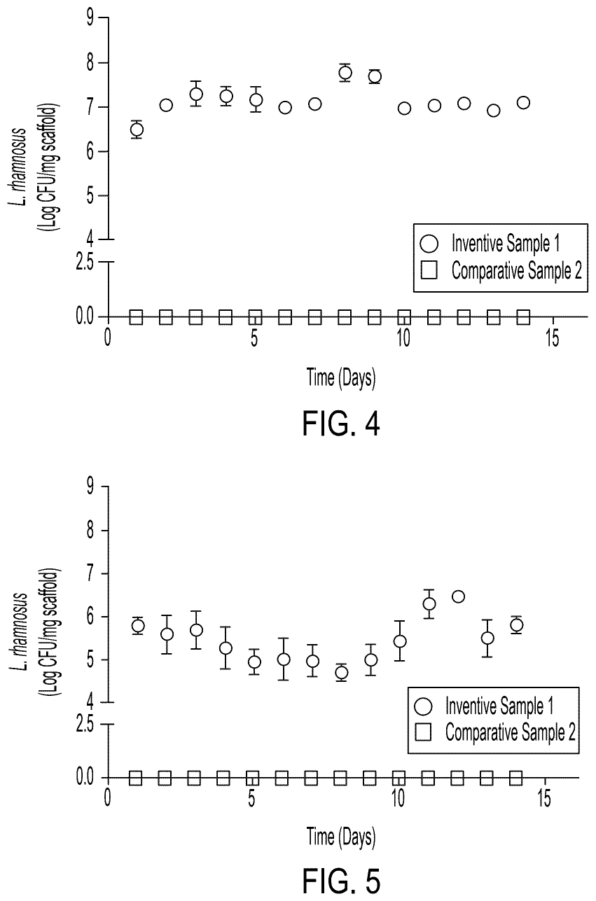

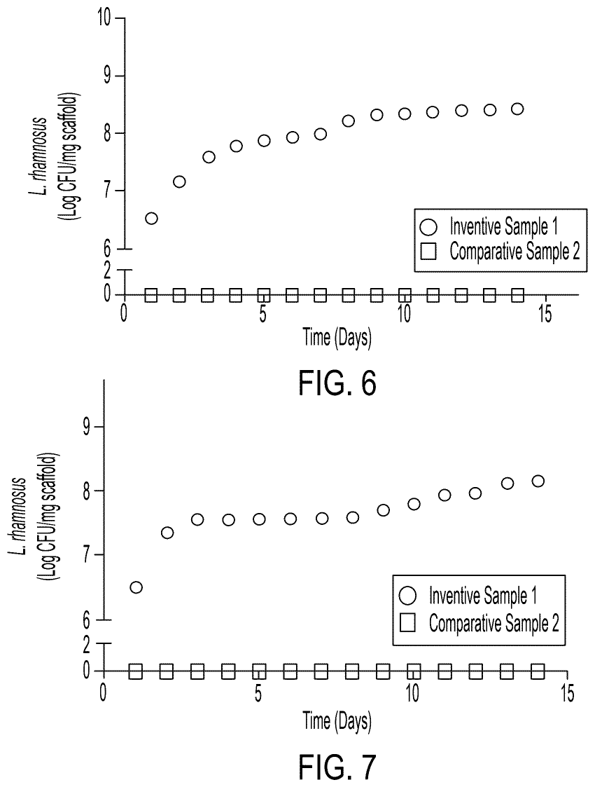

The viability of probiotic, L. rhamnosus in particular, in the test specimens was evaluated by measuring cumulative and daily recovery of live bacteria. The portion of an indwelling catheter present in the bladder is in contact with urine, while the length of the catheter through the urethra is in contact with the epithelium, each representing different cell nutrient environments. Therefore, representative samples of Inventive Sample 1 containing L. rhamnosus were evaluated for bacterial recovery from the samples placed in urine and MRS media. Specifically, daily recovery of L. rhamnosus due to bacterial release and proliferation was evaluated in MRS broth and in urine for 2 weeks with sampling each day. Inventive Sample 1 containing L. rhamnosus were initially weighed and then placed in 1.5 mL microcentrifuge tubes and incubated in 1 mL of MRS broth or urine at 37° C. with constant shaking at 150 rpm in aerobic conditions. The supernatant of each sample was removed daily for 1-14 days, and the sample supernatant was diluted by adding 20 μL of the sample to 180 μL of MRS broth or urine. Sample dilutions were plated on MRS agar plates and placed in an anaerobic chamber for 48 h. Plates were then evaluated for CFU counts. Prior to the next time point, scaffolds were washed four times in 5 mL of phosphate buffered saline (PBS), placed in 1 mL fresh MRS broth or urine, and incubated at 37° C. FIGS. 4 and 5 graphically illustrate the daily release of L. rhamnosus for each specimen at each time point for MRS and urine respectively. FIGS. 6 and 7 graphically illustrate the cumulative release of L. rhamnosus for each specimen at each time point for MRS and urine respectively. After 48 hours, L. rhamnosus daily recovery remained consistently greater than 10⁷ CFU/mg, and by 7 days, cumulative recovery reached 10⁸ CFU/mg in MRS. Additionally, L. rhamnosus recovery in urine reached a cumulative concentration of 2.2×10⁷ CFU/mg after 48 hours and 3.7×10⁷ CFU/mg by 7 days. Such testing results demonstrate continued viability and release of L. rhamnosus for an extended duration.

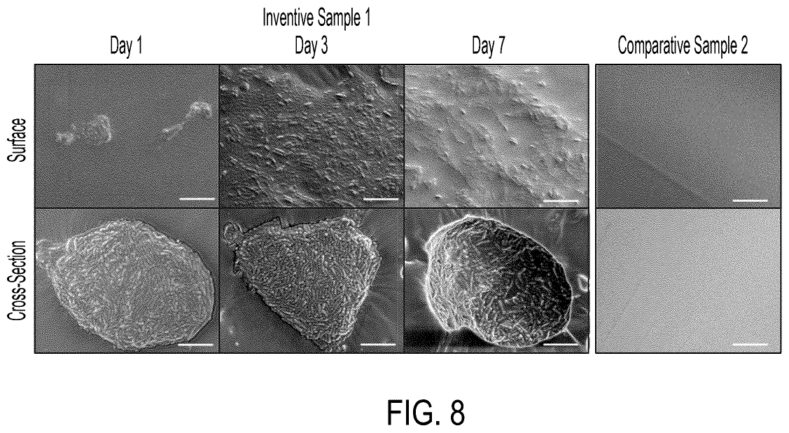

The sustained recovery of L. rhamnosus observed in FIGS. 4, 5, 6, and 7 suggest that the L. rhamnosus bacteria could be proliferating within, on the surface of the test specimens, or both. Scanning electron microscopy (SEM) was performed to visualize L. rhamnosus distribution and growth in and on representative examples of Inventive Sample 1 along with Comparative Sample 2 for comparison. Both surface and cross sections of Inventive Sample 1 and Comparative Sample 2 were characterized using scanning electron microscopy (SEM). The cross sections were immediately placed on carbon tape, sputter-coated with a layer of palladium/gold alloy (9.2 nm), and imaged using Apreo C LoVac Field Emission SEM (Thermo Scientific, Waltham, MA). The SEM imagery is provided in FIG. 8. Following incubation in MRS, the surface of Inventive Sample 1 was shown to have visually evident collections of bacteria that increased in size from 1 day to 3 and 7 days, consistent with the proliferation of surface-bound L. rhamnosus. Imaging of cross-sections of each sample also demonstrated intact L. rhamnosus distributed throughout the scaffold interior.

### Pathogenic Bacteria Growth Inhibition

As the end goal of the probiotic self-coating medical catheter is to minimize instances of CAUTI, it is desirable that the probiotic self-coating medical catheter be capable of preventing or correcting proliferation of a pathogenic bacteria or formation of a pathogenic bacteria film on the probiotic self-coating medical catheter. Accordingly, the ability for the test specimens to inhibit or kill pathogenic bacterial growth was determined. Uropathogenic E. coli (UPEC) growth was examined in the presence of Inventive Sample 1, Comparative Sample 2, and in supernatants collected from preincubation of Inventive Sample 1 and Comparative Sample 2.

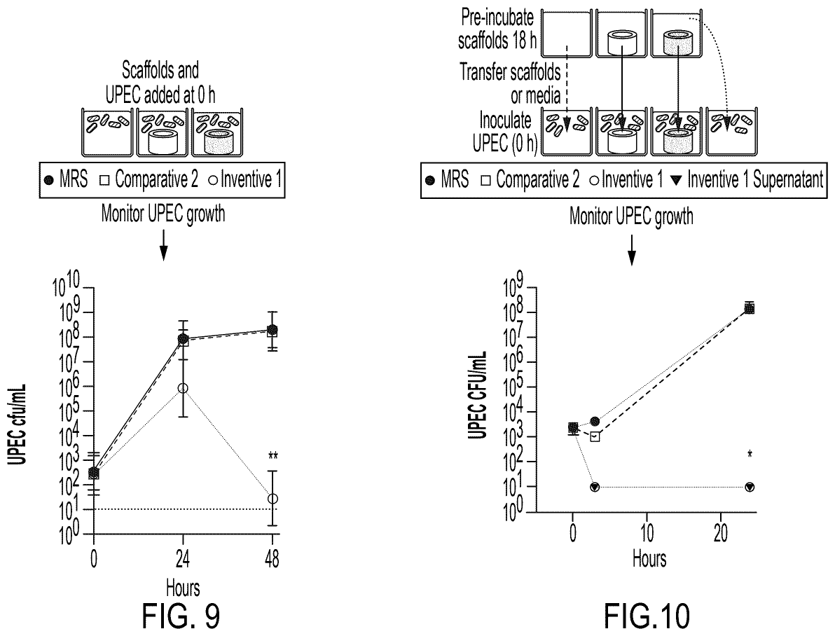

In a first test, UPEC was introduced into MRS media at the same time as representative samples of Inventive Sample 1 and Comparative Sample 2. Specifically, Inventive Sample 1 and Comparative Sample 2 were placed in 200 μL of MRS in a 96-well plate. Uropathogenic Escherichia coli (UPEC) strain UTI89 (with a kanamycin resistance cassette for selection) was diluted to 10⁵ CFU/mL in PBS from an 18 hour static Luria broth (LB) culture and 10 μL was added to each assay well. The plate was incubated statically at 37° C. with 5% CO2. An aliquot from each well was removed 24 and 48 hours later and UPEC CFU in the media were enumerated by dilution plating on LB+kan agar. At the 48 hour endpoint, Inventive Sample 1 and Comparative Sample 2 were removed from the media, gently rinsed in 1 mL of PBS in an Eppendorf tube, and then transferred to another tube with 1 mL of PBS. The tubes were sonicated for 5 min in a Bronson 200 bath from the surface of each of Inventive Sample 1 and Comparative Sample 2 and enumerated by dilution plating on LB+kan agar. The resulting enumeration of UPEC is illustrated in FIG. 9. FIG. 9 shows UPEC proliferated over the course of 48 hours in MRS alone as well as in Comparative Sample 2. In the presence of Inventive Sample 1 containing L. rhamnosus, UPEC displayed similar growth at 24 hours but was completely killed by 48 hours. This suggests that an initial incubation period was required for the L. rhamnosus in Inventive Sample 1 to begin exerting antimicrobial effects.

In a second test, UPEC was not introduced into MRS media at the same time as representative samples of Inventive Sample 1 and Comparative Sample 2. Instead, Inventive Sample 1 and Comparative Sample 2 were preincubated in 200 μL of MRS in a 96-well plate at 37° C. in 5% CO2. Inventive Sample 1 and Comparative Sample 2 were removed 18 hours later from the wells, washed with PBS (vortex in 1 mL in an Eppendorf tube), and transferred to new wells with fresh MRS. At such point, UPEC was added to each assay well with Inventive Sample 1 and Comparative Sample 2, fresh MRS, and to supernatant from the preincubation of Inventive Sample 1. The plate was incubated at 37° C. in 5% CO2. Aliquots were removed 3 and 24 h after the addition of UPEC and serially diluted and plated for CFU on LB+kan agar. The resulting enumeration of UPEC is illustrated in FIG. 10. FIG. 10 shows UPEC proliferated over the course of 24 hours in MRS alone as well as in preincubated Comparative Sample 2. In the presence of Inventive Sample 1 containing L. rhamnosus, UPEC did not grow but was instead killed by the 3 hour time. UPEC was also killed in supernatant collected from the preincubation of Inventive Sample 1.

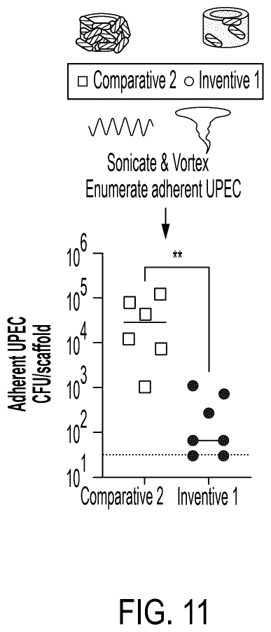

During CAUTI, uropathogens like UPEC not only grow in the bladder lumen but also bind to the surface of the catheter and form biofilms. Adherence of UPEC to Inventive Sample 1 and Comparative Sample 2 was quantified by washing, sonicating, and vortexing the samples to release the biofilm for CFU enumeration. The number of adherent UPEC was significantly lower on Inventive Sample 1 than on Comparative Sample 2. These results provide proof-of-concept that probiotic self-coating medical catheter are inhibitory against planktonic UPEC and against adherence to the silicone surface of the probiotic self-coating medical catheter. The comparison of adhered UPEC is illustrated in FIG. 11.

Having described various embodiments, it should be understood that the various aspects of the probiotic self-coating medical catheter and the method of making the same may be utilized in conjunction with various other aspects.

In a first aspect, the disclosure provides a method of making a probiotic self-coating medical catheter, the method comprising the steps of: preparing a probiotic bioink, wherein preparing the probiotic bioink comprises combining at least one probiotic with a silicone bioink at a weight ratio of 2:1 to 100:1; printing a three-dimensional tubular structure with the probiotic bioink; and heating the three-dimensional tubular structure to a temperature of up to 60° C. to cure the probiotic bioink through hydrosilylation of the silicone bioink to form the probiotic self-coating medical catheter, wherein the at least one probiotic is a non-pathogenic bacteria.

In a second aspect, the disclosure provides the method of the first aspect, in which the at least one probiotic is from the genus Lactobacillus.

In a third aspect, the disclosure provides the method of the first or second aspect, in which the at least one probiotic is selected from the group consisting of Lactobacillus rhamnosus, Lactobacillus acidophilus, Lactobacillus crispatus, Lactobacillus gasseri, Lactobacillus reuteri, Lactobacillus bulgaricus, Lactobacillus plantarum, Lactobacillus johnsonii, Lactobacillus paracasei, Lactobacillus casei, and Lactobacillus salivaris.

In a fourth aspect, the disclosure provides the method of any of the first through third aspects, in which the silicone bioink comprises polydimethylsiloxane.

In a fifth aspect, the disclosure provides the method of the first through third aspects, in which the silicone bioink comprises a copolymer of: i. vinyl-terminated poly(dimethylsiloxane) and vinyl, methyl-modified silica; and ii. methylhydrosiloxane-dimethylsiloxane copolymer, trimethylsiloxane-terminated.

In a sixth aspect, the disclosure provides the method of any of the first through fifth aspects, in which the at least one probiotic and the silicone bioink are combined at a weight ratio of 4:1 to 15:1 to form the probiotic bioink.

In a seventh aspect, the disclosure provides the method of any of the first through sixth aspects, in which preparing the probiotic bioink further comprises additionally combining at least one antibiotic with the at least one probiotic and the silicone bioink.

In an eighth aspect, the disclosure provides the method of any of the first through seventh aspects, in which the printing comprises extrusion, co-axial extrusion, fused deposition modelling, inkjet bio-printing, laser-assisted bioprinting, stereolithography, selective laser sintering (SLS), or combinations thereof.

In a ninth aspect, the disclosure provides the method of any of the first through eighth aspects, in which preparing the probiotic bioink further comprises additionally combining at least one composite filler with the at least one probiotic and the silicone bioink.

In a tenth aspect, the disclosure provides the method of any of the first through ninth aspects, in which the probiotic self-coating medical catheter is configured as a urinary catheter.

In an eleventh aspect, the disclosure provides a probiotic self-coating medical catheter, the catheter comprising a three-dimensional tubular structure having a central annulus, the tubular structure comprising a length, an outer diameter and an inner diameter with a wall thickness represented by the difference between the outer diameter and the inner diameter, wherein the tubular structure comprises a probiotic bioink in a cured state, the probiotic bioink comprising at least one probiotic and a silicone bioink at a weight ratio of 2:1 to 100:1

In a twelfth aspect, the disclosure provides the medical catheter of the eleventh aspect, in which the at least one probiotic is from the genus Lactobacillus.

In a thirteenth aspect, the disclosure provides the medical catheter of the eleventh or twelfth aspect, in which the at least one probiotic is selected from the group consisting of Lactobacillus rhamnosus, Lactobacillus acidophilus, Lactobacillus crispatus, Lactobacillus gasseri, Lactobacillus reuteri, Lactobacillus bulgaricus, Lactobacillus plantarum, Lactobacillus johnsonii, Lactobacillus paracasei, Lactobacillus casei, and Lactobacillus salivaris.

In a fourteenth aspect, the disclosure provides the medical catheter of any of the eleventh through thirteenth aspects, in which the silicone bioink comprises polydimethylsiloxane.

In a fifteenth aspect, the disclosure provides the medical catheter of any of the eleventh through thirteenth aspects, in which the silicone bioink comprises a copolymer of: i. vinyl-terminated poly(dimethylsiloxane) and vinyl, methyl-modified silica; and ii. methylhydrosiloxane-dimethylsiloxane copolymer, trimethylsiloxane-terminated.

In a sixteenth aspect, the disclosure provides the medical catheter of any of the eleventh through fifteenth aspects, in which the at least one probiotic and the silicone bioink are combined at a weight ratio of 4:1 to 15:1 to form the probiotic bioink.

In a seventeenth aspect, the disclosure provides the medical catheter of any of the eleventh through sixteenth aspects, in which the probiotic bioink further comprises at least one antibiotic.

In an eighteenth aspect, the disclosure provides the medical catheter of any of the eleventh through seventeenth aspects, in which the probiotic bioink further comprises at least one composite filler.

In a nineteenth aspect, the disclosure provides the medical catheter of any of the eleventh through eighteenth aspects, in which the probiotic self-coating medical catheter is configured as a urinary catheter.

In a twentieth aspect, the disclosure provides the medical catheter of any of the eleventh through eighteenth aspects, in which the probiotic self-coating medical catheter is configured as a nasogastric tube.

In a twenty-first aspect, the disclosure provides the medical catheter of any of the eleventh through eighteenth aspects, in which the probiotic self-coating medical catheter is configured as an intrapleural chest tube.

It should be apparent to those skilled in the art that various modifications and variations may be made to the embodiments described within without departing from the spirit and scope of the claimed subject matter. Thus, it is intended that the specification cover the modifications and variations of the various embodiments described within provided such modification and variations come within the scope of the appended claims and their equivalents.

As used throughout, the singular forms “a,” “an” and “the” include plural referents unless the context clearly dictates otherwise. Thus, for example, reference to “a” component includes aspects having two or more such components, unless the context clearly indicates otherwise.

It should be understood that any two quantitative values assigned to a property or measurement may constitute a range of that property or measurement, and all combinations of ranges formed from all stated quantitative values of a given property or measurement are contemplated in this disclosure.

As used here and in the appended claims, the words “comprise,” “has,” and “include” and all grammatical variations thereof are each intended to have an open, non-limiting meaning that does not exclude additional elements or steps.

Having described the subject matter of the present disclosure in detail and by reference to specific embodiments thereof, it is noted that the various details disclosed within should not be taken to imply that these details relate to elements that are essential components of the various embodiments described within, even in cases where a particular element is illustrated in each of the drawings that accompany the present description. Further, it should be apparent that modifications and variations are possible without departing from the scope of the present disclosure, including, but not limited to, embodiments defined in the appended claims. More specifically, although some aspects of the present disclosure are identified as particularly advantageous, it is contemplated that the present disclosure is not necessarily limited to these aspects.

What is claimed is:

1. A method of making a probiotic self-coating medical catheter, the method comprising the steps of:
preparing a probiotic bioink, wherein preparing the probiotic bioink comprises combining at least one probiotic with a silicone bioink at a weight ratio of 2:1 to 100:1;
printing a three-dimensional tubular structure with the probiotic bioink; and
heating the three-dimensional tubular structure to a temperature of up to 60° C. to cure the probiotic bioink through hydrosilylation of the silicone bioink to form the probiotic self-coating medical catheter,
wherein the at least one probiotic is a non-pathogenic bacteria.

2. The method of claim 1, wherein the at least one probiotic is from the genus Lactobacillus.

3. The method of claim 1, wherein the at least one probiotic is selected from the group consisting of Lactobacillus rhamnosus, Lactobacillus acidophilus, Lactobacillus crispatus, Lactobacillus gasseri, Lactobacillus reuteri, Lactobacillus bulgaricus, Lactobacillus plantarum, Lactobacillus johnsonii, Lactobacillus paracasei, Lactobacillus casei, and Lactobacillus salivaris.

4. The method of claim 1, wherein the silicone bioink comprises polydimethylsiloxane.

5. The method of claim 1, wherein the silicone bioink comprises a copolymer of:
i. vinyl-terminated poly(dimethylsiloxane) and vinyl, methyl-modified silica; and
ii. methylhydrosiloxane-dimethylsiloxane copolymer, trimethylsiloxane-terminated.

6. The method of claim 1, wherein the at least one probiotic and the silicone bioink are combined at a weight ratio of 4:1 to 15:1 to form the probiotic bioink.

7. The method of claim 1, wherein preparing the probiotic bioink further comprises additionally combining at least one antibiotic with the at least one probiotic and the silicone bioink.

8. The method of claim 1, wherein the printing comprises extrusion, co-axial extrusion, fused deposition modelling, inkjet bio-printing, laser-assisted bioprinting, stereolithography, selective laser sintering (SLS), or combinations thereof.

9. The method of claim 1, wherein preparing the probiotic bioink further comprises additionally combining at least one composite filler with the at least one probiotic and the silicone bioink.

10. The method of claim 1, wherein the probiotic self-coating medical catheter is configured as a urinary catheter.

11. A probiotic self-coating medical catheter, the catheter comprising
a three-dimensional tubular structure having a central annulus, the tubular structure comprising a length, an outer diameter and an inner diameter with a wall thickness represented by the difference between the outer diameter and the inner diameter,
wherein the tubular structure comprises a probiotic bioink in a cured state, the probiotic bioink comprising at least one probiotic and a silicone bioink at a weight ratio of 2:1 to 100:1.

12. The probiotic self-coating medical catheter of claim 11, wherein the at least one probiotic is from the genus Lactobacillus.

13. The probiotic self-coating medical catheter of claim 11, wherein the at least one probiotic is selected from the group consisting of Lactobacillus rhamnosus, Lactobacillus acidophilus, Lactobacillus crispatus, Lactobacillus gasseri, Lactobacillus reuteri, Lactobacillus bulgaricus, Lactobacillus plantarum, Lactobacillus johnsonii, Lactobacillus paracasei, Lactobacillus casei, and Lactobacillus salivaris.

14. The probiotic self-coating medical catheter of claim 11, wherein the silicone bioink comprises polydimethylsiloxane.

15. The probiotic self-coating medical catheter of claim 11, wherein the silicone bioink comprises a copolymer of:
i. vinyl-terminated poly(dimethylsiloxane) and vinyl, methyl-modified silica; and
ii. methylhydrosiloxane-dimethylsiloxane copolymer, trimethylsiloxane-terminated.

16. The probiotic self-coating medical catheter of claim 11, wherein the at least one probiotic and the silicone bioink are combined at a weight ratio of 4:1 to 15:1 to form the probiotic bioink.

17. The probiotic self-coating medical catheter of claim 11, wherein the probiotic bioink further comprises at least one antibiotic.

18. The probiotic self-coating medical catheter of claim 11, wherein the probiotic bioink further comprises at least one composite filler.

19. The probiotic self-coating medical catheter of claim 11, wherein the probiotic self-coating medical catheter is configured as a urinary catheter.

20. The probiotic self-coating medical catheter of claim 11, wherein the probiotic self-coating medical catheter is configured as a nasogastric tube.

21. The probiotic self-coating medical catheter of claim 11, wherein the probiotic self-coating medical catheter is configured as an intrapleural chest tube.
